(19) United States
(12) Patent Application Publication
(10) Pub. No.: US 2024/0341586 A1
(43) Pub. Date: Oct. 17, 2024

(54) SELF-GUIDED EYE EXAMINATION SYSTEM

(71) Applicant: EYECHEQ INC., Miami, FL (US)

(72) Inventors: Ali Khoshnevis, Miami, FL (US); Rashid Taher, Miami, FL (US); James E. Barber, Cleveland, OH (US); David J. Boll, Cleveland, OH (US); David Michael Pehar, Cleveland, OH (US); Peter R. Sords, Cleveland, OH (US); Robert Vystrcil, Cleveland, OH (US); Sloan Zimmerman, Cleveland, OH (US)

(21) Appl. No.: 18/298,557

(22) Filed: Apr. 11, 2023

(51) Int. Cl. A61B 3/032; A61B 3/00; A61B 3/14

(52) U.S. Cl. CPC A61B 3/032; A61B 3/0033; A61B 3/0041; A61B 3/14

(57) ABSTRACT

A self-guided eye examination system includes an eye examination interface module mounted on a linear actuator that is configured to move the eye examination interface module along a vertical axis. The eye examination interface module includes a housing having at least one window on a front face of the housing, a frame inside of the housing, and at least one eye examination device mounted to the frame and accessible from an outside of the housing. The at least one eye examination device is configured to examine at least one aspect of a user's health. At least one graphical user interface is configured to guide the user in operating the self-guided eye examination system without assistance from an attendant. A processor is configured to cause the eye examination device to examine an aspect of the user's health and provide feedback to the user.

### TECHNICAL FIELD

The present disclosure relates generally to ophthalmologic testing systems, and more particularly to a self-guided eye examination system.

### BACKGROUND

Nearly 95% of blindness is preventable when detected early. Accordingly, it is advantageous for patients to obtain regular eye examinations to maintain an awareness of the health of their eyes and seek remedial or corrective medical measures when necessary. Most eye examinations, however, require a visit to an ophthalmologist's office, where an ophthalmologist can test the patient's eyes using various specialized cameras and testing equipment, and interpret the results of the tests to give a diagnosis. Many patients, however, may find scheduling, travelling to and from, and participating in such a visit inconvenient and difficult. Accordingly, many patients do not keep up on regular visits to an ophthalmologist and therefore may miss early detection of eye health related issues that can cause blindness or other visual disabilities.

### SUMMARY

Accordingly, there is a need for more convenient and easy access to eye examination systems, such as those that do not require a patient to schedule a visit to an ophthalmologist's office. The eye examination system described herein is a self-guided eye examination system that can be operated by a user, without the assistance of an attendant or ophthalmologist, and results in a convenient and positive user experience. The self-guided eye examination system is configured to perform an eye examination and provide feedback to the user regarding a result of the eye examination. For example, the self-guided eye examination system may refer the user to a health care provider for any necessary follow-up. The self-guided eye examination kiosk is modular and compact in structure, such that it can be transported and implemented in a convenient location, such as in retail outlets, pharmacies, and grocery stores, where users are more likely to visit in their day-to-day schedule. In this manner, a user is more likely to use the self-guided eye examination system to examine the health of their eyes, allowing early detection of disease and assisting users on their health care journey in an effort to avoid preventable blindness.

According to an aspect of the present disclosure, a self-guided eye examination system includes an eye examination interface module mounted on a linear actuator. The linear actuator is configured to move the eye examination interface module along a vertical axis. The eye examination interface module includes a housing including at least one window on a front face of the housing, a frame inside of the housing, and at least one eye examination device mounted to the frame inside of the housing and accessible from an outside of the housing at the front face of the housing through the at least one window. The at least one eye examination device is configured to examine at least one aspect of a user's health. The self-guided eye examination system also includes at least one graphical user interface configured to guide the user in operating the self-guided eye examination system without assistance from an attendant, and a processor configured to cause the at least one eye examination device to examine the at least one aspect of the user's health and provide feedback to the user on the at least one graphical user interface based on at least one result of the at least one aspect of the user's health.

According to an embodiment of one or more paragraph(s) of this disclosure, the eye examination interface module is manually movable by the user manually engaging the linear actuator to move the eye examination interface module along the vertical axis to ensure proper alignment of the eye examination interface module relative to the user during operation of the self-guided eye examination system.

According to an embodiment of one or more paragraph(s) of this disclosure, the housing of the eye examination interface module includes at least one handle for manually engaging the linear actuator and manually moving the eye examination interface module along the vertical axis to ensure proper alignment of the eye examination interface module relative to the user during operation of the self-guided eye examination system.

According to an embodiment of one or more paragraph(s) of this disclosure, the processor is configured to variably engage the linear actuator to move the eye examination interface module along the vertical axis to ensure proper alignment of the eye examination interface module relative to the user during operation of the self-guided eye examination system.

According to an embodiment of one or more paragraph(s) of this disclosure, the self-guided eye examination system further includes an artificial intelligence camera configured to detect at least one feature of the user and, based on the at least one feature of the user, cause the processor to automatically engage the linear actuator to move the eye examination interface module along the vertical axis to ensure proper alignment of the eye examination interface module relative to the user during operation of the self-guided eye examination system.

According to an embodiment of one or more paragraph(s) of this disclosure, the graphical user interface includes a user height input and, based on the user height input, causes the processor to automatically engage the linear actuator to move the eye examination interface module along the vertical axis to ensure proper alignment of the eye examination interface module relative to the user during operation of the self-guided eye examination system.

According to an embodiment of one or more paragraph(s) of this disclosure, the self-guided eye examination system further includes a user controlled activator accessible from an outside of the housing and coupled to the processor, the user controlled activator configured to, based on activation by the user, cause the processor to engage the linear actuator to move the eye examination interface module along the vertical axis to ensure proper alignment of the eye examination interface module relative to the user during operation of the self-guided eye examination system.

According to an embodiment of one or more paragraph(s) of this disclosure, the at least one eye examination device includes at least one of a retinal camera, an autorefractor, and a visual acuity testing device, and the at least one aspect of a user's health respectively includes at least one of an image of the user's retina, a refractive error of the user, and a visual acuity of the user.

According to an embodiment of one or more paragraph(s) of this disclosure, the visual acuity testing device includes a convoluted tunnel having a series of straight segments. The convoluted tunnel has a light emitting diode or liquid crystal display at a first end of the convoluted tunnel and a visual acuity display at a second end of the convoluted tunnel. The light emitting diode or liquid crystal display is configured to emit a visual acuity symbol into the convoluted tunnel. The visual acuity testing device also includes a mirror in each corner of adjoining straight segments of the convoluted tunnel, each mirror being configured to reflect the visual acuity symbol emitted by the light emitting diode or liquid crystal display such that the visual acuity symbol is reflected to the visual acuity display. The visual acuity display is visually accessible from an outside of the housing of the eye examination interface module at a front face of the housing and is configured to display the visual acuity symbol emitted by the light emitting diode or liquid crystal display.

According to an embodiment of one or more paragraph(s) of this disclosure, the convoluted tunnel forms a path between the light emitting diode or liquid crystal display and the visual acuity display having a distance in a range of 149.20 to 161.90 centimeters.

According to an embodiment of one or more paragraph(s) of this disclosure, the self-guided eye examination system further includes an artificial intelligence camera configured to detect a position of the user relative to the visual acuity display. The processor is configured to instruct, via the at least the graphical user interface, the user to move relative to the visual acuity display, based on the detected position of the user, to ensure that the user is positioned in a range of 38.10 to 50.80 centimeters of the visual acuity display.

According to an embodiment of one or more paragraph(s) of this disclosure, the artificial intelligence camera is configured to detect a posture of the user, and wherein the processor is configured to instruct, via the graphical user interface, the user to adjust the posture based on the detected posture to ensure compliance with the visual acuity testing device.

According to an embodiment of one or more paragraph(s) of this disclosure, the at least one eye examination device includes at least one shield for protecting at least a part of the at least one eye examination device when not in use.

According to an embodiment of one or more paragraph(s) of this disclosure, wherein the at least one shield includes at least one bellows for covering a gap between the at least one eye examination device and a periphery of the at least one window in the housing.

According to an embodiment of one or more paragraph(s) of this disclosure, the at least one shield includes a retractable shield movable between a retracted position and a shielding position.

According to an embodiment of one or more paragraph(s) of this disclosure, the retractable shield includes a motor, at least one gear rotatable by the motor, and a flexible door having a series of gear holes in which the at least one gear is configured to engage the flexible door. When the motor rotates the at least on gear in a first direction, the at least one gear is configured to engage the series of gear holes in the flexible door and coil the flexible door around the at least one gear to move the retractable shield from the shielding position to the retracted position. When the motor rotates the at least one gear in a second direction, opposite the first direction, the at least one gear is configured to engage the series of gear holes in the flexible door and uncoil the flexible door from around the at least one gear to move the retractable door from the retracted position to the shielding position.

According to an embodiment of one or more paragraph(s) of this disclosure, the housing of the eye examination interface module includes a proximity sensor on a bottom face of the housing. The proximity sensor is configured to detect a presence of an object within a predetermined distance from the bottom face of the housing and cause the processor to prevent the linear actuator from moving the eye examination interface module along the vertical axis into contact with the object.

According to an embodiment of one or more paragraph(s) of this disclosure, the housing of the eye examination interface module includes a bumper on a bottom face of the housing.

According to an embodiment of one or more paragraph(s) of this disclosure, the bumper includes a contact sensor configured to detect contact of the bumper with an object.

According to an embodiment of one or more paragraph(s) of this disclosure, the linear actuator is mounted on a stand-alone kiosk.

According to an embodiment of one or more paragraph(s) of this disclosure, the kiosk includes a back frame on which the linear actuator is mounted, a canopy attached to a top end of the back frame and cantilevered over the eye examination interface module, and two side walls attached to a bottom end of the back frame and extending out perpendicularly from the back frame. The back frame, the canopy and the two side walls define an operating space in which the eye examination interface module resides.

According to an embodiment of one or more paragraph(s) of this disclosure, at least one of the two side walls includes at least one handrail.

According to an embodiment of one or more paragraph(s) of this disclosure, at least one of the two side walls includes an integrated storage unit.

According to an embodiment of one or more paragraph(s) of this disclosure, the integrated storage unit includes at least one of a sanitizing wipe dispenser and a trash can.

According to an embodiment of one or more paragraph(s) of this disclosure, the self-guided eye examination system further includes a plurality of casters on a bottom of the back frame and the two side walls.

According to an embodiment of one or more paragraph(s) of this disclosure, the linear actuator is mounted on a tabletop stand.

According to an embodiment of one or more paragraph(s) of this disclosure, the linear actuator is mounted to a ceiling.

According to an embodiment of one or more paragraph(s) of this disclosure, the linear actuator is mounted to a wall.

According to an embodiment of one or more paragraph(s) of this disclosure, the at least one user interface is provided on the front face of the housing of the eye examination interface module.

According to an embodiment of one or more paragraph(s) of this disclosure, the at least one user interface is provided on a mobile device.

According to an embodiment of one or more paragraph(s) of this disclosure, the eye examination interface module further includes a display screen on the front face of the housing.

According to an embodiment of one or more paragraph(s) of this disclosure, the eye examination interface module further includes at least one speaker on the front face of the housing.

According to an embodiment of one or more paragraph(s) of this disclosure, the processor is further configured to communicate the at least one result of the at least one aspect of the user's health to a remote practitioner for assessment.

According to an embodiment of one or more paragraph(s) of this disclosure, the processor is configured to provide feedback to the user based on the at least one result of the at least one aspect of the user's health in the form of a referral to a remote practitioner.

The following description and the annexed drawings set forth in detail certain illustrative embodiments described in this disclosure. These embodiments are indicative, however, of but a few of the various ways in which the principles of this disclosure may be employed. Other objects, advantages and novel features will become apparent from the following detailed description when considered in conjunction with the drawings.

### BRIEF DESCRIPTION OF DRAWINGS

The annexed drawings show various aspects of the disclosure.

FIG. 1 is a front perspective view of a self-guided eye examination system.

FIG. 2 is a rear perspective view of a self-guided eye examination system.

FIG. 3 is a schematic diagram of a self-guided eye examination system.

FIG. 4 is a front perspective view of an eye examination device in a self-guided eye examination system.

FIG. 5 is a front perspective view of another eye examination device in a self-guided eye examination system.

FIG. 6 is a front perspective view of another eye examination device in a self-guided eye examination system.

FIG. 7 is a rear perspective view of the eye examination device of FIG. 6 in a self-guided eye examination system.

FIG. 8 is a front perspective view of an eye examination device in a self-guided eye examination system.

FIG. 9 is a side cross-sectional view of the eye examination device of FIG. 8 in a self-guided eye examination system.

FIG. 10 is an exploded view of a shield for the eye examination device of FIGS. 8 and 9 in a self-guided eye examination system.

FIG. 11 is a front perspective view of another self-guided eye examination system.

FIG. 12 is a bottom perspective view of a kiosk for a self-guided eye examination system.

### DETAILED DESCRIPTION

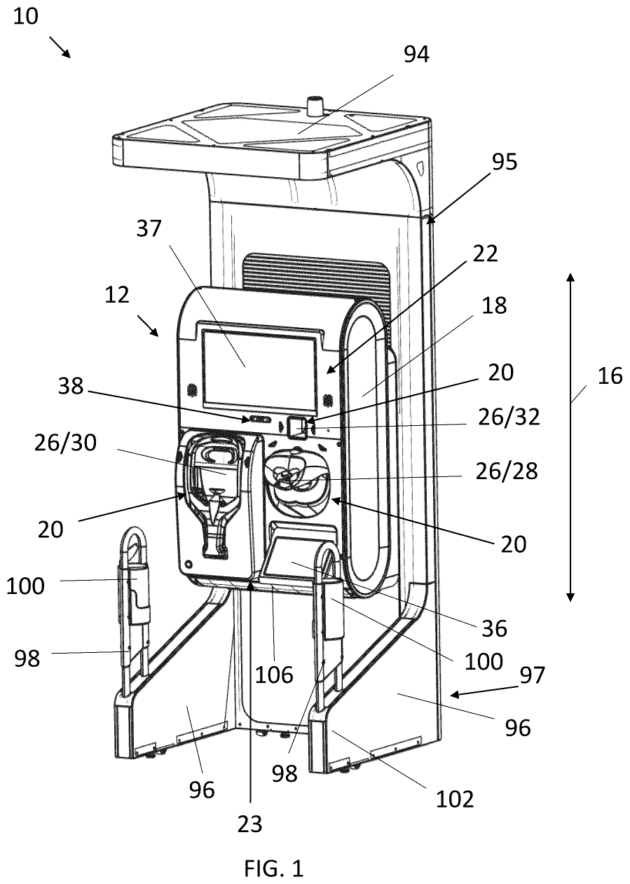

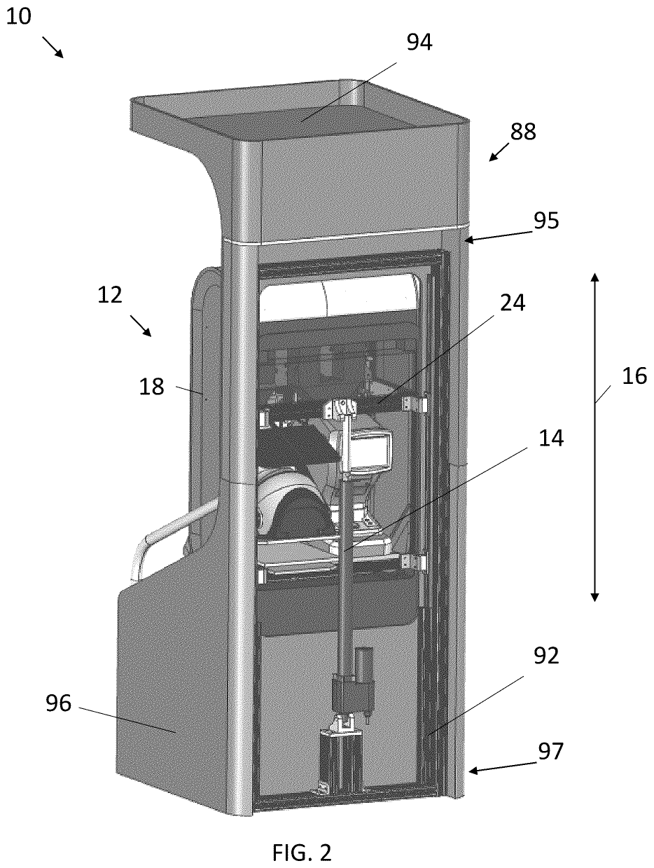

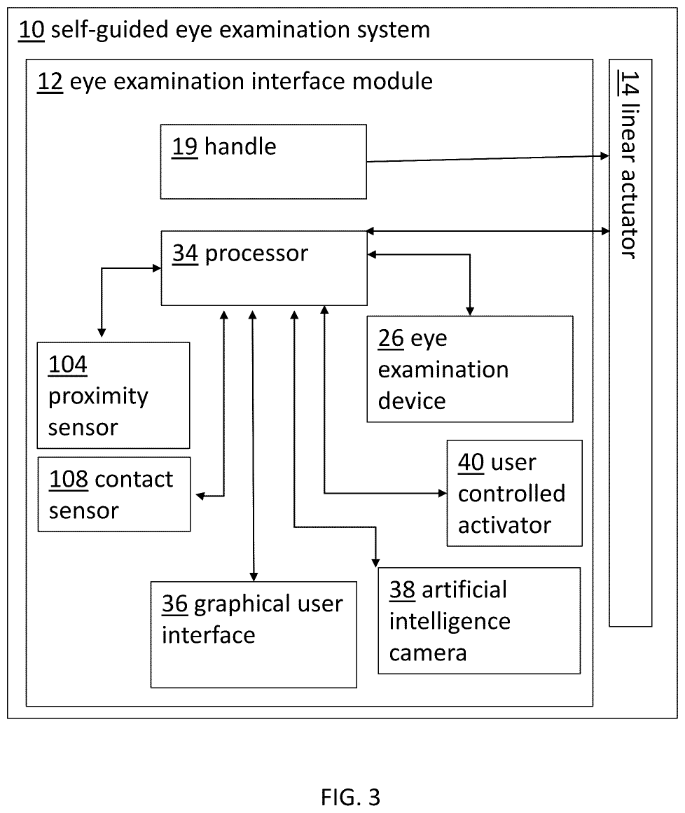

With initial reference to FIGS. 1-3, a self-guided eye examination system 10 is depicted. FIG. 1 shows the self-guided eye examination system 10 from a front perspective view and FIG. 2 shows the self-guided eye examination system 10 from a rear perspective view. FIG. 3 depicts a schematic of the self-guided eye examination system 10. The self-guided eye examination system 10 includes an eye examination interface module 12 mounted on a linear actuator 14, which is configured to move the eye examination interface module 12 along a vertical axis 16 to accommodate various heights of a user using the self-guided eye examination system 10. For example, the eye examination interface module 12 may be moved along the vertical axis 16 to accommodate a user in a seated position (e.g., in a chair or a wheelchair) or in a standing position in a variety of heights. The movement of the eye examination interface module 12 along the vertical axis 16 will ensure proper alignment of the eye examination interface module 12 with respect to the user.

The eye examination interface module 12 includes a housing 18 having at least one window 20 on a front face 22 of the housing 18. A frame 24 is inside of the housing 18 and at least on eye examination device 26 is mounted to the frame 24 inside of the housing 18. The linear actuator 14 is mounted to the frame 24, specifically, so that the linear actuator 14 moves the frame 24 along the vertical axis 16, thereby moving the housing 18 and the at least one eye examination device 26 commensurately along the vertical axis 16.

The at least one eye examination device 26 is accessible from an outside of the housing 18 at the front face 22 of the housing 18 through the at least one window 20 for usage by the user, as depicted in FIG. 1, and may extend out of a back opening of the housing 18, as depicted in FIG. 2. The at least one eye examination device 26 is configured to examine at least one aspect of a user's health during operation of the self-guided eye examination system 10. For example, the at least one eye examination device 26 may include at least one of a retinal camera 28, an autorefractor 30, and a visual acuity testing device 32. The at least one aspect of a user's health may therefore respectively include at least one of an image of the user's retina (obtained by the retinal camera 28), a refractive error of the user (obtained by the autorefractor 30), and a visual acuity of the user (obtained by the visual acuity testing device 32). It is understood, however, that there may be additional or alternative eye examination devices incorporated into the eye examination interface module 12 and that the foregoing devices are provided as non-limiting examples. It is also understood that other health examination devices may be incorporated into the eye examination interface module 12, such as a blood pressure monitor, a weight scale and an electrocardiogram, as non-limiting examples.

Turning to FIG. 3, the eye examination interface module 12 also includes a processor 34 configured to cause the at least one eye examination device 26 to examine the at least one aspect of the user's health. Upon examining the at least one aspect of the user's health, the at least one eye examination device 26 communicates at least one result of the at least one aspect of the user's health back to the processor 34. The processor 34 is then configured to provide feedback to the user based on at least one result of the at least one aspect of the user's health. Specifically, the eye examination interface module 12 also includes at least one graphical user interface 36 configured to guide the user in operating the self-guided eye examination system without assistance from an attendant or healthcare professional. The processor 34 may therefore provide the feedback to the user via the at least one graphical user interface 36. For example, the processor 34 may provide feedback to the user based on the at least one result of the at least one aspect of the user's health in the form of a referral to a remote practitioner. For example, the feedback may be the user's visual acuity, the user's refractive error, and/or a picture of the user's retinal scan. If the examination is conducted in a clinical setting, the feedback may also include a diagnosis. However, the processor 34 may further be configured to communicate the at least one result to a remote practitioner for assessment and diagnosis, such that if and when the user follows up with the referred remote practitioner, the remote practitioner will have the at least one result and will be able to provide appropriate care to the user based on the at least one result. The processor may be configured to communicate the at least one result to a remote practitioner via, for example, a hardware internet connection (e.g., with an ethernet connection) or a wireless or cellular internet connection.

The at least one graphical user interface 36 may be provided on the front face 22 of the housing 18 of the eye examination interface module 12, as depicted in FIG. 1, or may additionally or alternatively be provided on a mobile device of the user, such as a smartphone or a tablet. The eye examination interface module 12 may also include a display screen 37 that is configured to display various retail advertisements or other graphical displays, and may also be coupled to the processor 34 for providing feedback to the user. The eye examination interface module 12 may also include at least one speaker on the front face 22 of the housing 18 for emitting audio feedback to the user during operation of the self-guided eye examination system 10.

The eye examination interface module 12 may be manually movable by the user to accommodate a height of the user. Specifically, the user may manually engage the linear actuator 14 by for example grabbing the housing 18 of the eye examination interface module 12 and moving the housing 18 of the eye examination interface module 12 to move the eye examination interface module 12 along the vertical axis 16. The housing 18 may include at least one handle 19 for assisting the user to grab and move the housing 18 and therefore engage the linear actuator 14 to move the eye examination interface module 12 along the vertical axis 16. In this manner, the user may manually move the eye examination interface module 12 to ensure proper alignment of the eye examination interface module 12 relative to the user during operation of the self-guided eye examination system 10.

In another embodiment, the processor 34 is configured to variably engage the linear actuator 14 to automatically move the eye examination interface module 12 along the vertical axis 16 to ensure proper alignment of the eye examination interface module 12 relative to the user during operation of the self-guided eye examination system 10, to accommodate a height of the user. For example, the self-guided eye examination system 10 may include an artificial intelligence camera 38 on the front face 22 of the housing 18, the artificial intelligence camera 38 being configured to detect at least one feature of the user and, based on the detected at least one feature of the user, cause the processor 34 to automatically engage the linear actuator 14 to move the eye examination interface module 12 along the vertical axis 16 to ensure proper alignment of the eye examination interface module 12 relative the user during operation of the self-guided eye examination system 10. Additionally or alternatively, the at least one graphical user interface 36 may include a user height input. Based on the user height input, input into the at least one graphical user interface 36 by the user, the at least one graphical user interface 36 causes the processor 34 to automatically engage the linear actuator 14 to move the eye examination interface module 12 along the vertical axis 16 to ensure proper alignment of the eye examination interface module 12 relative to the user during operation of the self-guided eye examination system 10. Further, additionally or alternatively the self-guided eye examination system 10 may include a user-controlled activator 40 accessible from an outside of the housing 18 and coupled to the processor 34. The user-controlled activator 40 may be configured to, based on activation by the user, cause the processor 34 to engage the linear actuator 14 to move the eye examination interface module 12 along the vertical axis 16 to ensure proper alignment of the eye examination interface module 12 relative to the user during operation of the self-guided eye examination system 10. For example, the user-controlled activator 40 may include a plurality of directional buttons or a toggle for controlling the direction of movement of the eye examination interface module 12.

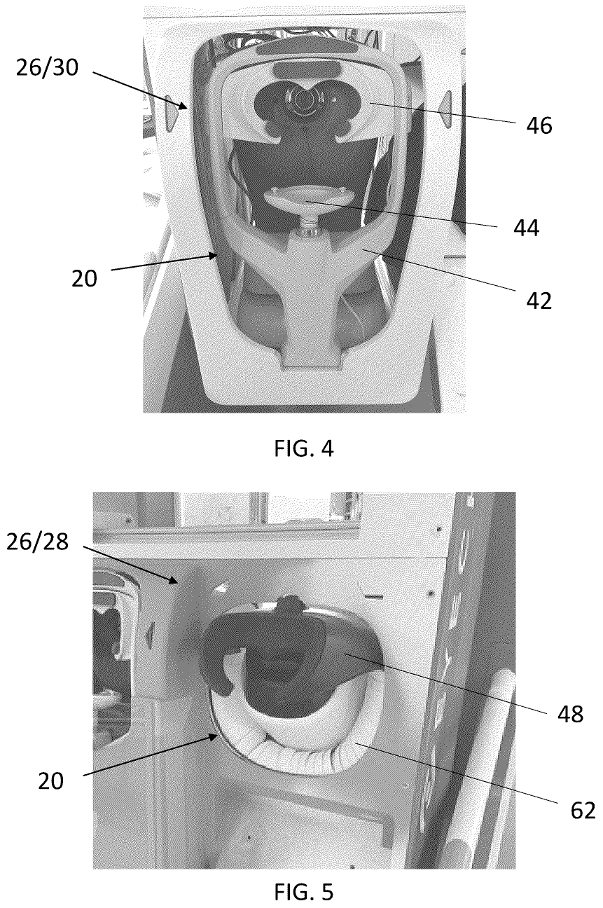

With reference to FIG. 4, the autorefractor 30 is depicted as an exemplary eye examination device 26 of the eye examination interface module 12. The autorefractor 30 is configured to evaluate the refractive error of the user by shining a light into the user's eye, measuring how it changes as it bounces off the back of the eye (the user's retina). The autorefractor 30 then shows the user an image that moves in and out of focus and as reflections are made, several measurements determine when the eye is properly focused. The autorefractor 30 therefore has a face frame 42 for securing the user's face in place relative to the autorefractor 30 so that the autorefractor 30 can take such measurements. The face frame 42 includes a chin rest 44 on which the user is to place their chin and an eye frame 46 for securing the user's eyes in a proper position for the autorefractor 30 to take such measurements. Although the autorefractor 30 is largely housed within the housing 18 of the eye examination interface module 12, the face frame 42 of the autorefractor 30 is accessible from the outside of the housing 18 at the front face 22 of the housing 18 through the at least one window 20 such that the face frame 42 of the autorefractor 30 is accessible to the user.

FIG. 5 depicts the retinal camera 28 as another exemplary eye examination device 26 of the eye examination interface module 12. The retinal camera 28 is configured to obtain an image of the user's retina for the evaluation and screening of retinopathy, or damage to the blood vessels of the light sensitive tissue of the retina. The retinal camera 28 includes an eye support 48 for receiving and supporting the user's eyes during operation of the retinal camera 28. Although the retinal camera 28 is largely housed within the housing 18 of the eye examination interface module 12, the eye support 48 of the retinal camera 28 is accessible from the outside of the housing 18 at the front face 22 of the housing 18 through the at least one window 20 such that the eye support 48 of the retinal camera 28 is accessible to the user.

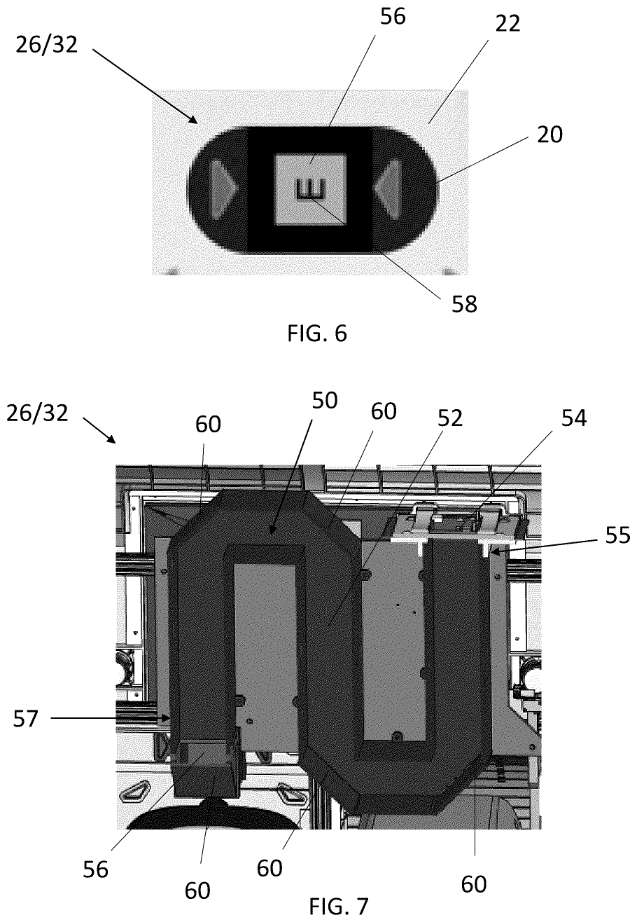

FIGS. 6-7 depict the visual acuity testing device 32 as another exemplary eye examination device 26 of the eye examination interface module 12. The visual acuity testing device 32 is configured to determine the visual acuity of the user. The visual acuity testing device 32 includes a convoluted tunnel 50 having a series of straight segments 52 to form a compact path traversing a distance in a range of 149.20 to 161.90 centimeters, for example 154.28 centimeters, to allow for proper visual acuity testing. The convoluted tunnel 50 includes a liquid crystal display or light emitting diode display 54 at a first end 55 of the convoluted tunnel 50 and a visual acuity display 56 at a second end 57 of the convoluted tunnel 50. The display 54 is configured to emit a visual acuity symbol 58 into the convoluted tunnel 50 at the first end 55 of the convoluted tunnel 50. A mirror 60 is provided in each corner of adjoining straight segments 52 of the convoluted tunnel 50, as well as where the final straight segment 52 adjoins the visual acuity display 56 such that each mirror 60 is configured to reflect the visual acuity symbol 58 emitted by the display 54 through the path of the convoluted tunnel 50 to the visual acuity display 56 at the second end 57 of the convoluted tunnel 50. In this manner, the visual acuity symbol 58 emitted by the display 54 at the first end 55 of the convoluted tunnel 50 may be reflected onto the visual acuity display 56 at the second end of the convoluted tunnel 50. Although the visual acuity testing device 32, including the convoluted tunnel 50 and the display 54, is largely housed within the housing 18 of the eye examination interface module 12, the visual acuity display 56 of the visual acuity testing device 32 is visually accessible from the outside of the housing 18 at the front face 22 of the housing 18 through the at least one window 20 such that the visual acuity display 56 is configured to display the visual acuity symbol 58 emitted by the display 54 to the user during operation of the visual acuity testing device 32. It will be understood that the convoluted tunnel 50 having a series of straight segments 32, liquid crystal display or light emitting diode display 54, and mirrors 60 is provided as a non-limiting example of simulating distance. For example, other methods and technologies, such as those using lenses or parabolic mirrors, may be used to simulate a variety of distances. Additionally, it will be understood that other types of light projection may be used in place of or in addition to the liquid crystal display or light emitting diode display 54.

As briefly mentioned earlier, the eye examination interface module 12 may include an artificial intelligence camera 38. The artificial intelligence camera 38 may be additionally configured to detect a position of the user relative to the visual acuity display 56. Based on the detected position of the user relative to the visual acuity display 56, the processor 34 may be configured to instruct, via the at least one graphical user interface 36 or the at least one speaker, the user to move themselves relative to the visual acuity display 56, so that they are positioned in a range of 38.10 to 50.80 centimeters, for example 45.72 centimeters, from the visual acuity display 56 to ensure proper testing by the visual acuity testing device 32. The artificial intelligence camera 38 may also detect a posture of the user, including a position of the user's extremities relative to the user's body. For example, for proper visual acuity testing, a user must cover one of their eyes when testing the other eye. Accordingly, to ensure compliance with the requirements for proper visual acuity testing, the artificial intelligence camera 38 may be configured to detect whether the user is adequately covering a respective eye during operation of the visual acuity testing device 32. Based on the detected posture of the user, the processor 34 may be configured to instruct, via the at least one graphical user interface 36 or the at least one speaker, the user to adjust their posture to ensure compliance with the visual acuity testing device 32. Specifically, if the posture of the user is not compliant with the visual acuity testing device 32 (e.g., if the user is not covering one of their eyes), the processor 34 may be configured to instruct the user to adjust their posture to ensure compliance (e.g., to cover one of their eyes) before causing the visual acuity testing device 32 to examine the user's visual acuity.

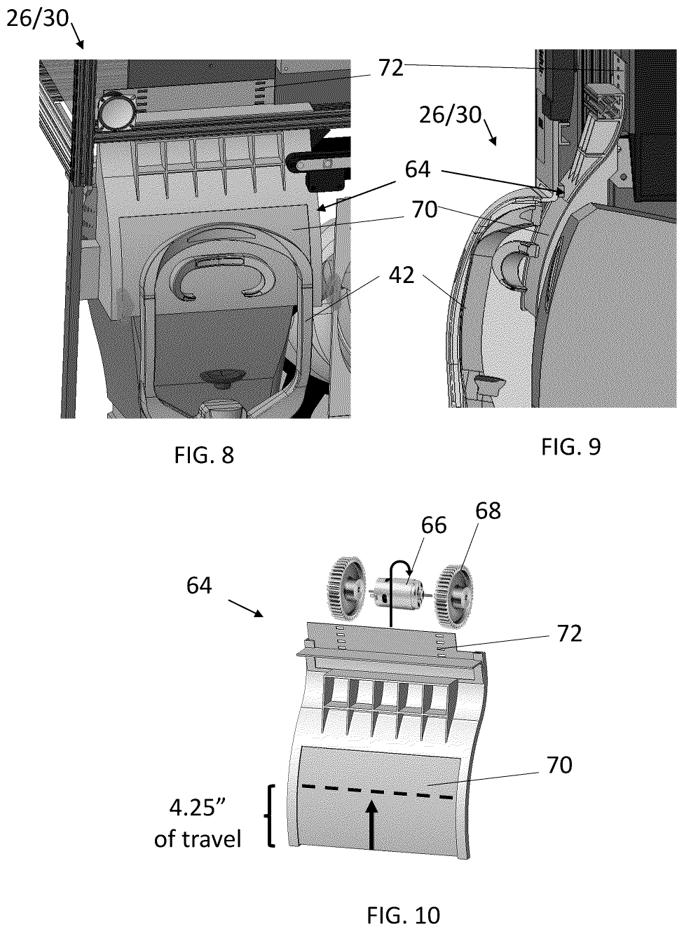

One or more of the at least one eye examination device 26 (e.g., the autorefractor 30, the retinal camera 28 and/or the visual acuity testing device 32) may include a shield for protecting the at least one eye examination device 26 when not in use. For example, the at least one eye examination device 26 may include a bellows 62, as depicted for the retinal camera 28 in FIG. 5, for covering a gap between the at least one eye examination device 26 and a periphery of the at least one window 20. Additionally or alternatively, with reference to the autorefractor 30 depicted in FIGS. 8-10, the at least one eye examination device 26 may include a retractable shield 64. The retractable shield 64 is movable between a retracted position and a shielding position between the face frame 42 and the rest of the autorefractor 30 housed within the housing 18. As depicted, the retractable shield 64 may include a motor 66 configured to rotate at least one gear 68, and a flexible door 70 engageable with the at least one gear 68. As the motor 66 rotates the at least one gear 68, the at least one gear 68 engages with the flexible door 70, for example, by engaging a series of gear holes 72 in the flexible door 70, to roll the flexible door 70 around the gears 86 and move the retractable shield 64 from the shielding position to the retracted position. To move the retractable shield 64 from the retracted position to the shielding position, the motor 66 is configured to rotate the gears 68 in the opposite direction to unroll the flexible door 70 from around the gears 68. In this manner, when the autorefractor 30 is not in use, the processor 34 is configured to instruct the retractable shield 64 to move to the shielding position, and when the user wishes to use the autorefractor 30, the processor 34 is configured to instruct the retractable shield 64 to move to the retracted position.

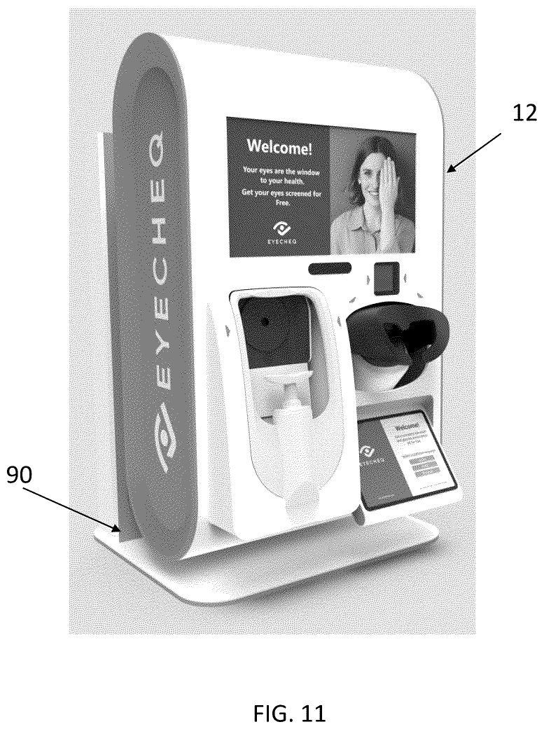

Referring back to FIGS. 1 and 2, the linear actuator 14 may be mounted on a stand-alone kiosk 88, therefore integrating the eye examination interface module 12 with the stand-alone kiosk 88. Alternatively, the linear actuator 14 may be mounted on a tabletop stand 90, as depicted in FIG. 11, or may be mounted to a wall or suspended from a ceiling. As best seen in FIG. 2, the kiosk 88 includes a back frame 92 on which the linear actuator 14 is mounted. A canopy 94 is attached to a top end 95 of the back frame 92 and is cantilevered over the eye examination interface module 12 mounted in front of the back frame 92. The canopy 94 is configured to create a controlled lighting environment for operation of the eye examination interface module 12. The kiosk 88 also includes two side walls 96 attached to a bottom end 97 of the back frame 92 and extending out perpendicularly from the back frame 92. The back frame 92, the canopy 94 and the two side walls 96 therefore define an operating space in which the eye examination interface module 12 resides and in which the user can stand or sit while operating the eye examination interface module 12. At least one of the two side walls 96 may include at least one handrail 98 that the user can grasp to stabilize themselves and orient themselves relative to the eye examination interface module 12. A front piece 102 of each of the side walls 96 may be removably attached to the rest of the side walls 96, such that each front piece 102 may be removed while transporting the kiosk 88, for example through doorways. Any surface of the kiosk 88, including the canopy 94, the side walls 96 and the back frame 92 may be used for retail advertisements or other graphic displays.

At least one of the two side walls 96 and/or the at least one handrail 98 may include at least one integrated storage unit 100. For example, the at least one integrated storage unit 100 may include at least one of a sanitizing wipe dispenser and a trash can. For example, as depicted in FIG. 1, one of the handrails 98 may include an integrated sanitizing wipe dispenser, and the other of the handrails 98 may include an integrated trash can for disposing of the sanitizing wipes after use.

The eye examination interface module 12 may have a proximity sensor 104 located on a bottom face 23 of the housing 18. The proximity sensor 104 is configured to detect a presence of an object within a predetermined distance from the bottom face 23 of the housing 18 and cause the processor 34 to prevent the linear actuator 14 from moving the eye examination interface module 12 along the vertical axis 16 into contact with the object. In this way, the self-guided eye examination system 10 is configured to avoid injuring or bumping into a user during operation of the self-guided eye examination system 10 as the eye examination interface module 12 is moved. Additionally or alternatively, the housing of the eye examination interface module may include a bumper 106 on the bottom face 23 of the housing 18 in an event that the proximity sensor 104 malfunctions. The bumper 106 is made of a soft material that will mitigate any impact of the eye examination interface module 12 contacting an object when moving. The bumper 106 may include a contact sensor 108 that is configured to detect contact with an object and cause the processor 34 to stop the linear actuator 14 from moving the eye examination interface module 12.

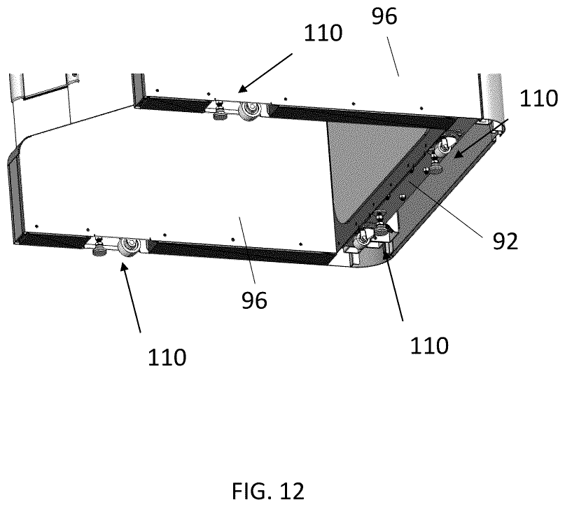

The kiosk 88 is configured to be easily movable and transportable. With reference to FIG. 12, for example, the kiosk 88 may include a plurality of casters 110 on a bottom of the back frame 92 and side walls 96. Each of the plurality of casters 110 are configured to be deployed so that the kiosk 88 may roll on the casters 110, and be retracted so that the kiosk 88 may sit stationary on the floor. For example, at least some of the casters 110 may be quick release (step-down type) casters that may be deployed or retracted so that the kiosk 88 may be easily locked in place or released to be moved.

In any implementation of the self-guided eye examination system 10 (e.g., whether in the kiosk 88, tabletop stand 90, wall mounted or ceiling mounted), a user is able to operate the self-guided eye examination system 10 to obtain one or more examinations of at least one aspect of their health, without the assistance of an attendant or health practitioner. Specifically, the self-guided eye examination system 10 may include a non-transitory computer readable medium storing program code, which when executed by the processor 34, guides the user, via the graphical user interface 36 and/or at least one speaker, in operating the self-guided eye examination system 10. The non-transitory computer readable medium may also be configured to store user examination results and create a health profile for the user. In this manner, a user may return to the self-guided eye examination system 10 for routine or sequential examinations to track the progress of their eye health over time, and access their personalized medical health record via the graphical user interface 36. For example, when a user begins operating the self-guided eye examination system 10, the user may access their personalized medical health record and user profile by entering a username and password into the graphical user interface 36. Alternatively, the user may access their personalized medical health record via password-less registration, such as via short message service (SMS) and/or a QR code. The non-transitory computer readable medium may also be configured to adaptively screen and sort users operating the self-guided eye examination system 10 based on a triage protocol into various categories for ongoing user engagement and may update a user's status accordingly.

During operation of the self-guided eye examination system 10, the non-transitory computer readable medium may be configured to query the user via the graphical user interface 36 about various health-related questions, such as symptoms or other health updates. The non-transitory computer readable medium may then offer one or more examination using the at least one eye examination device 26 to the user. The user may then select the one or more examination and the non-transitory computer readable medium is configured to guide the user in operating the at least one eye examination device 26, as briefly described earlier. Upon completion of the one or more examination using the at least one eye examination device 26, the non-transitory computer readable medium is configured to provide feedback, via the graphical user interface 36 to the user and/or send at least one result to a remote practitioner, as described earlier.

Although the above disclosure has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments. In addition, while a particular feature may have been described above with respect to only one or more of several illustrated embodiments, such feature may be combined with one or more other features of the other embodiments, as may be desired and advantageous for any given or particular application.

1. A self-guided eye examination system, comprising:
an eye examination interface module mounted on a linear actuator, the linear actuator configured to move the eye examination interface module along a vertical axis, the eye examination interface module including:
a housing including at least one window on a front face of the housing;
a frame inside of the housing; and
at least one eye examination device mounted to the frame inside of the housing and accessible from an outside of the housing at the front face of the housing through the at least one window, the at least one eye examination device configured to examine at least one aspect of a user's health;
at least one graphical user interface configured to guide the user in operating the self-guided eye examination system without assistance from an attendant; and
a processor configured to cause the at least one eye examination device to examine the at least one aspect of the user's health and provide feedback to the user on the at least one graphical user interface based on at least one result of the at least one aspect of the user's health.

2. The self-guided eye examination system according to claim 1, wherein the eye examination interface module is manually movable by the user manually engaging the linear actuator to move the eye examination interface module along the vertical axis to ensure proper alignment of the eye examination interface module relative to the user during operation of the self-guided eye examination system.

3. The self-guided eye examination system according to claim 1, wherein the housing of the eye examination interface module includes at least one handle for manually engaging the linear actuator and manually moving the eye examination interface module along the vertical axis to ensure proper alignment of the eye examination interface module relative to the user during operation of the self-guided eye examination system.

4. The self-guided eye examination system according to claim 1, wherein the processor is configured to variably engage the linear actuator to move the eye examination interface module along the vertical axis to ensure proper alignment of the eye examination interface module relative to the user during operation of the self-guided eye examination system.

5. The self-guided eye examination system according to claim 4, further comprising an artificial intelligence camera configured to detect at least one feature of the user and, based on the at least one feature of the user, cause the processor to automatically engage the linear actuator to move the eye examination interface module along the vertical axis to ensure proper alignment of the eye examination interface module relative to the user during operation of the self-guided eye examination system.

6. The self-guided eye examination system according to claim 4, wherein the graphical user interface includes a user height input and, based on the user height input, causes the processor to automatically engage the linear actuator to move the eye examination interface module along the vertical axis to ensure proper alignment of the eye examination interface module relative to the user during operation of the self-guided eye examination system.

7. The self-guided eye examination system according to claim 4, further comprising a user controlled activator accessible from an outside of the housing and coupled to the processor, the user controlled activator configured to, based on activation by the user, cause the processor to engage the linear actuator to move the eye examination interface module along the vertical axis to ensure proper alignment of the eye examination interface module relative to the user during operation of the self-guided eye examination system.

8. The self-guided eye examination system according to claim 1, wherein the at least one eye examination device includes at least one of a retinal camera, an autorefractor, and a visual acuity testing device, and the at least one aspect of a user's health respectively includes at least one of an image of the user's retina, a refractive error of the user, and a visual acuity of the user.

9. The self-guided eye examination system according to claim 8, wherein the visual acuity testing device includes:
a convoluted tunnel having a series of straight segments, the convoluted tunnel having a light emitting diode or liquid crystal display at a first end of the convoluted tunnel and a visual acuity display at a second end of the convoluted tunnel, the light emitting diode or liquid crystal display configured to emit a visual acuity symbol into the convoluted tunnel;
a mirror in each corner of adjoining straight segments of the convoluted tunnel, each mirror being configured to reflect the visual acuity symbol emitted by the light emitting diode or liquid crystal display such that the visual acuity symbol is reflected to the visual acuity display;
wherein the visual acuity display is visually accessible from an outside of the housing of the eye examination interface module at a front face of the housing and is configured to display the visual acuity symbol emitted by the light emitting diode or liquid crystal display.

10. The self-guided eye examination system according to claim 9, wherein the convoluted tunnel forms a path between the light emitting diode or liquid crystal display and the visual acuity display having a distance in a range of 149.20 to 161.90 centimeters.

11. The self-guided eye examination system according to claim 9, further comprising an artificial intelligence camera configured to detect a position of the user relative to the visual acuity display, wherein the processor is configured to instruct, via the at least the graphical user interface, the user to move relative to the visual acuity display, based on the detected position of the user, to ensure that the user is positioned in a range of 38.10 to 50.80 centimeters of the visual acuity display.

12. The self-guided eye examination system according to claim 11, wherein the artificial intelligence camera is configured to detect a posture of the user, and wherein the processor is configured to instruct, via the graphical user interface, the user to adjust the posture based on the detected posture to ensure compliance with the visual acuity testing device.

13. The self-guided eye examination system according to claim 1, wherein the at least one eye examination device includes at least one shield for protecting at least a part of the at least one eye examination device when not in use.

14. The self-guided eye examination system according to claim 13, wherein the at least one shield includes at least one bellows for covering a gap between the at least one eye examination device and a periphery of the at least one window in the housing.

15. The self-guided eye examination system according to claim 13, wherein the at least one shield includes a retractable shield movable between a retracted position and a shielding position.

16. The self-guided eye examination system according to claim 15, wherein the retractable shield includes:
a motor;
at least one gear rotatable by the motor;
a flexible door having a series of gear holes in which the at least one gear is configured to engage the flexible door;
wherein when the motor rotates the at least on gear in a first direction, the at least one gear is configured to engage the series of gear holes in the flexible door and coil the flexible door around the at least one gear to move the retractable shield from the shielding position to the retracted position; and
wherein when the motor rotates the at least one gear in a second direction, opposite the first direction, the at least one gear is configured to engage the series of gear holes in the flexible door and uncoil the flexible door from around the at least one gear to move the retractable door from the retracted position to the shielding position.

17. The self-guided eye examination system according to claim 1, wherein the housing of the eye examination interface module includes a proximity sensor on a bottom face of the housing, the proximity sensor being configured to detect a presence of an object within a predetermined distance from the bottom face of the housing and cause the processor to prevent the linear actuator from moving the eye examination interface module along the vertical axis into contact with the object.

18. The self-guided eye examination system according to claim 1, wherein the housing of the eye examination interface module includes a bumper on a bottom face of the housing.

19. The self-guided eye examination system according to claim 18, wherein the bumper includes a contact sensor configured to detect contact of the bumper with an object.

20. The self-guided eye examination system according to claim 1, wherein the linear actuator is mounted on a stand-alone kiosk.

21. The self-guided eye examination system according to claim 20, wherein the kiosk includes:
a back frame on which the linear actuator is mounted;
a canopy attached to a top end of the back frame and cantilevered over the eye examination interface module; and
two side walls attached to a bottom end of the back frame and extending out perpendicularly from the back frame,
wherein the back frame, the canopy and the two side walls define an operating space in which the eye examination interface module resides.

22. The self-guided eye examination system according to claim 21, wherein at least one of the two side walls includes at least one handrail.

23. The self-guided eye examination system according to claim 21, wherein at least one of the two side walls includes an integrated storage unit.

24. The self-guided eye examination system according to claim 23, wherein the integrated storage unit includes at least one of a sanitizing wipe dispenser and a trash can.

25. The self-guided eye examination system according to claim 20, further comprising a plurality of casters on a bottom of the back frame and the two side walls.

26. The self-guided eye examination system according to claim 1, wherein the linear actuator is mounted on a tabletop stand.

27. The self-guided eye examination system according to claim 1, wherein the linear actuator is mounted to a ceiling.

28. The self-guided eye examination system according to claim 1, wherein the linear actuator is mounted to a wall.

29. The self-guided eye examination system according to claim 1, wherein the at least one user interface is provided on the front face of the housing of the eye examination interface module.

30. The self-guided eye examination system according to claim 1, wherein the at least one user interface is provided on a mobile device.

31. The self-guided eye examination system according to claim 1, wherein the eye examination interface module further includes a display screen on the front face of the housing.

32. The self-guided eye examination system according to claim 1, wherein the eye examination interface module further includes at least one speaker on the front face of the housing.

33. The self-guided eye examination system according to claim 1, wherein the processor is further configured to communicate the at least one result of the at least one aspect of the user's health to a remote practitioner for assessment.

34. The self-guided eye examination system according to claim 1, wherein the processor is configured to provide feedback to the user based on the at least one result of the at least one aspect of the user's health in the form of a referral to a remote practitioner.
